(19) United States
(12) Patent Application Publication
(10) Pub. No.: US 2009/0105368 A1
(43) Pub. Date: Apr. 23, 2009

(54) TIME-CONTROLLED INTRAORAL FILM FORMER SYSTEM FOR INTRAORAL USE

(75) Inventors: Byoung I. Suh, Oak Brook, IL (US); Jin Whan Lee, Naperville, IL (US)

Correspondence Address: ICE MILLER LLP, ONE AMERICAN SQUARE, SUITE 3100, INDIANAPOLIS, IN, 46282-0200, US

(73) Assignee: Bisco, Inc., Schaumburg, IL (US)

(21) Appl. No.: 12/254,591

(22) Filed: Oct. 20, 2008

Related U.S. Application Data
(60) Provisional application No. 60/981,392, filed on Oct. 19, 2007.

(51) Int. Cl. A61K 6/08

(52) U.S. Cl. 523/116

(57) ABSTRACT

A water soluble polymer or biodegradable polymer operable to act as a release or separator between a prepared and sealed dentin surface and a temporary restoration.

### PRIORITY

This application claims priority to U.S. Provisional Patent Application Ser. No. 60/981,392, filed on Oct. 19, 2007, entitled “TIME-CONTROLLED INTRAORAL FILM FORMER SYSTEM FOR INTRAORAL USE”, the contents of which is incorporated herein by reference.

### BACKGROUND

In certain dental restorative procedures, including indirect dental restorations, a tooth or other intraoral surface is prepared, and then temporarily covered while the indirect restoration is being prepared (sometimes referred to as the “provisional phase” of indirect restorative procedures). In most instances, the patient is discharged after the provisional phase while the indirect restoration is prepared, with the patient returning to the practitioner in a time frame of approximately one to four weeks to replace the temporary covering with the completed indirect restoration (sometimes referred to as the “cementation phase” of indirect restorative procedures). While previous protocol called for dentin sealing to take place during the cementation phase or after the impression of the prepared surface was made in the provisional phase, there has been some indication that immediately sealing the dentin with a dentin bonding agent after surface preparation during the provisional phase (known as “immediate dentin sealing” or “IDS”) may improve bonding procedures by improving the dentin/bonding agent interface and providing a stronger surface upon which to later place the restoration.

While the IDS procedure provides a strong bond between the dentin and the dentin bonding agent, care must be taken not to weaken that bond by affixing the temporary restoration on the dentin bonding agent so strongly that it must be forcibly removed in a way that fractures the temporary restoration and leaves portions of it affixed to the prepared surface, or in such a way that the bonding agent surface is compromised. In an attempt to solve this issue, water-resistant chemicals such as wax or petroleum jelly or water-miscible glycerine have been used as a separating or releasing agent to coat the prepared surface prior to affixing the temporary restoration. However, the above-mentioned water-resistant materials are difficult to apply, and can be difficult to thoroughly remove from the prepared surface during the cementation phase, thereby potentially compromising the bond between the prepared surface and the permanent restoration. Further, complete removal of materials such as wax, petroleum jelly, or glycerine may require solvents that are toxic or unpleasant to a patient, and are not desirable to be used intraorally.

### BRIEF DESCRIPTION OF THE DRAWINGS

FIG. 1 is a chart comparing relative shear bond strength in a pushout test, comparing the relative ease of releasing a temporary restoration placed over a separating agent.

FIG. 2 is a bar graph showing the shear bond strength in a pullout test, comparing formulations of separating agents.

### SUMMARY

The present application relates to separating agents to prevent temporary restorations from adhering to an adhesive coating on a tooth surface.

In at least one embodiment, a separating agent composition comprises a first non-ionic portion comprising a polymer comprising a hydrophobic and hydrophilic portion and a second portion comprising an alcohol. Optionally, the first portion of the separating agent comprises approximately 10% to approximately 50% by weight of the separating agent. Further optionally, the first portion of the separating agent comprises approximately 35% to approximately 42% by weight of the separating agent. As yet another option, the first portion of the separating agent may be polyoxypropylene-polyoxyethylene block copolymer and/or poly(lactic-co-glycolic acid). Further optionally, the second portion of the separating agent is ethanol. As another option, the separating agent may comprise water.

In at least one other embodiment, a temporary dental restoration adhesion system comprises a first dental resin operable to be applied to a prepared dental surface to seal said prepared surface, the dental resin comprising a hydrophobic portion; a separating agent operable to coat a surface formed by the dental resin to act as a separation layer, the separating agent comprising a first non-ionic portion comprising a polymer comprising a hydrophobic and hydrophilic portion, and the separating agent further comprising a second portion comprising an alcohol; and a second dental resin operable to be applied over the separation layer, the second dental resin comprising a hydrophobic portion such that the second dental resin is operable to form a bond with the hydrophobic portion of the separating agent. Optionally, the separating agent further comprises water. As yet another option, the second dental resin is operable to form a removable bond with the separation layer such that the second dental resin, when cured, may be removed from the separation layer with less than 0.7 MPa pullout force. As a further option, the second dental resin is operable to form a removable bond with the separation layer such that the second dental resin, when cured, may be removed from the separation layer with less than 0.5 MPa pullout force. In at least one other optional embodiment, the first portion of the separating agent comprises approximately 10% to approximately 50% of the separating agent by weight. As yet at least one other optional embodiment, the first portion of the separating agent comprises approximately 35% to approximately 42% of the separating agent by weight.

Further, it will be appreciated that in at least one optional embodiment, the first non-ionic portion of the separating agent is selected from the group consisting of polyoxypropylene-polyoxyethylene block copolymer and poly(lactic-co-glycolic acid). Further, the first non-ionic portion of the separating agent may be comprised of microparticles or nanoparticles. In one optional embodiment, the separating agent is ethanol. Optionally, in the temporary dental restoration adhesion system the separating agent further comprises water.

In at least one other embodiment, a temporary dental restoration adhesion system comprises a first dental resin operable to be applied to a prepared dental surface to seal said prepared dental surface, the dental resin comprising a hydrophobic portion; a separating agent operable to coat a surface formed by the dental resin to act as a separation layer, the separating agent comprising a first portion comprising polyoxypropylene-polyoxyethylene block copolymer having a hydrophobic and hydrophilic portion, and the separating agent further comprising a second portion comprising ethanol, wherein the first portion comprises approximately 10% to approximately 50% of the separating agent by weight; and a second dental resin operable to be applied over the separation layer, the second dental resin comprising a hydrophobic portion such that the second dental resin is operable to form a bond with the hydrophobic portion of the separating agent. As an option, the polyoxypropylene-polyoxyethylene block copolymer may be dispersed throughout the separating agent as nanoparticles.

### DETAILED DESCRIPTION

The present application relates to separating agents to prevent temporary restorations from adhering to an adhesive coating on a tooth surface.

According to one embodiment of the present application, a water-soluble polymer or biodegradable polymer composition is utilized as a film to produce a barrier between a prepared tooth surface and a resin-based or resin adhered temporary restoration or prosthesis. Optionally, the water-soluble polymer or biodegradable polymer is a hydrogel. In one exemplary embodiment according to the present application, the water-soluble polymer or biodegradable polymer is combined with micro- or nano-particles comprising inorganic compounds. The micro- or nano-particles may be dissolved or dispersed in acetone, ethyl alcohol, and/or a water milieu to obtain proper mixture with the water-soluble or biodegradable polymer.

### EXAMPLES

A. Water-Soluble Polymer Utilizing PVP and Ethanol

According to one exemplary embodiment of the present application, the water-soluble polymer or biodegradable polymer composition comprises poly(N-vinylpyrrolidone) (“PVP”), and may optionally comprise ethanol, such as an HPLC grade ethanol (“EtOH”). According to certain embodiments of the present application, a water-soluble polymer composition comprising PVP and EtOH solution comprises approximately 1%, approximately 2%, approximately 3%-6%, approximately 6%-10%, approximately 10%-20%, approximately 20%-30%, or approximately 30%-40% PVP by weight, approximately 0.1%-2% agar, with the balance comprising EtOH. In one exemplary embodiment, the water-soluble polymer comprises 10% PVP, 0.2% polysaccharide agar, 0.1% 2-FDCBL01 blue dye, with the balance comprising ethanol or ethanol and water. Further optionally, a silica-gel or a nano-sealed silicate is added to a concentration of less than 50% by weight of the water-soluble polymer. Further, a neutralizing base may be added to the water-soluble polymer to neutralize any polyacrylic acid that may form lumps within the polymer.

In at least one embodiment, the water-soluble or biodegradable polymer further comprises a pigment. For example, the pigment such as 2-FDCBL01, a blue water soluble dye is added to the water-soluble or biodegradable polymer at a concentration of about 0.1%-2% by weight.

B. Water-Soluble Polymer Utilizing PVP and Fumed Silica

According to one exemplary embodiment of the present application, the water-soluble polymer or biodegradable polymer composition comprises poly(N-vinylpyrrolidone) (“PVP”), an HPLC grade ethanol (“EtOH”), and a fumed silica such as Aerosil R972 (available from Evonik Industries). According to certain embodiments of the present application, a water-soluble polymer composition comprising PVP and EtOH solution comprises approximately 30%-50% PVP by weight, approximately 1-10% fumed silica by weight, with the balance comprising EtOH. In yet another exemplary embodiment, the water-soluble polymer discussed immediately heretofore further comprises polyethylene glycol (“PEG”) in a concentration of about 5%-25% by weight, and more specifically about 8%-12% by weight. Further optionally, the water-soluble polymer comprises a dye such as 2-FDCBL01 in a concentration of about 0.1% by weight. As yet another option, the water-soluble gel comprises a fluorosurfactant such as 3M Fluorad 1-FC4432 in a concentration of about 0.1-0.5% by weight, and more specifically, about 0.2% by weight.

C. Water-Soluble Polymer Utilizing Agar and Ethanol

According to one exemplary embodiment of the present application, the water-soluble polymer or biodegradable polymer composition comprises poly(N-vinylpyrrolidone) (“PVP”), and may optionally comprise ethanol, such as an HPLC grade ethanol (“EtOH”). According to certain embodiments of the present application, a water-soluble polymer composition comprising PVP and EtOH solution comprises approximately 1%, approximately 2%, approximately 3%-6%, approximately 6%-10%, approximately 10%-20%, approximately 20%-30%, or approximately 30%-40% PVP by weight, with the balance comprising EtOH. In yet another exemplary embodiment, the water-soluble polymer further comprises Carbopol ETD 2691 (available from Noveon, Inc., hereinafter “Carbopol”), According to certain exemplary embodiments, the ratio of PVP to Carbopol to is about 100:0, about 75:25, about 50:50, about 25:75, or about 0:100 by weight, with the PVP and Carbopol combination comprising approximately 8%-10%, approximately 10%-12%, approximately 12%-15%, approximately 15%-20% by weight of the water-soluble polymer, and the balance of the water-soluble polymer comprising ethanol. Further optionally, a silica-gel or a nano-sealed silicate is added in place of, or in addition to, Carbopol, with the silica-gel or nano-sealed silicate present in a concentration of less than 50% by weight of the water-soluble polymer. Further, a neutralizing base may be added to the water-soluble polymer to neutralize any polyacrylic acid that may form lumps within the polymer. Alternatively, a nonionic hydrogel such as HPMC or HEC may be used, or a higher concentration of PVP may be used to induce viscous dispersion of the PVP in the ethanol solution.

In at least one embodiment, the water-soluble or biodegradable polymer further comprises a pigment. For example, the pigment such as 2-FDCBL01, a blue water soluble dye is added to the water-soluble or biodegradable polymer at a concentration of about 0.1%-2% by weight.

D. Water-Soluble Polymer Utilizing Ethylhydroxyethyl Cellulose and Ethanol

According to one exemplary embodiment of the present application, the water-soluble polymer or biodegradable polymer composition comprises ethylhydroxyethyl cellulose (“EHEC”), and may optionally comprise ethanol, such as an HPLC grade ethanol (“EtOH”). According to certain embodiments of the present application, a water-soluble polymer composition comprising EHEC and EtOH solution comprises approximately 1%, approximately 2%, approximately 2%-4%, approximately 5%, approximately 5%-6%, approximately 6%-10%, approximately 10%, approximately 10%-20%, approximately 20%-30%, or approximately 30%-40% EHEC by weight, with the balance comprising EtOH. In one exemplary embodiment, the water-soluble polymer comprises 10% EHEC, with the balance comprising ethanol or ethanol and water. Further optionally 0.1%-2.0% by weight of 2-FDCBL01 blue dye may be added to the water soluble polymer.

E. Biodegradable Polymer Utilizing PLGA and Acetone

According to one exemplary embodiment of the present application, the biodegradable polymer composition comprises poly(lactic-co-glycolic acid) (“PLGA”) and HPLC grade acetone. According to certain embodiments of the present application, a biodegradable polymer composition comprising PLGA and acetone comprises approximately 5%-25% PLGA by weight or 8%-10% by weight, with the balance comprising acetone. In yet another exemplary embodiment, the biodegradable polymer discussed immediately heretofore further comprises a dye such as 2-FDCBL01 in a concentration of about 0.1% by weight. As yet another option, the water-soluble gel comprises a fluorosurfactant such as 3M Fluorad 1-FC4432 (“FC”) in a concentration of about 0.1-0.5% by weight, and more specifically, about 0.2% by weight.

F. Biodegradable Polymer Utilizing PLGA and EHEC

According to one exemplary embodiment of the present application, the biodegradable polymer composition comprises poly(lactic-co-glycolic acid) (“PLGA”), ethylhydroxyethyl cellulose (“EHEC”), and HPLC grade acetone. According to certain embodiments of the present application, a biodegradable polymer composition comprising PLGA, EHEC and acetone comprises approximately 2%-25% PLGA by weight, 2%-25% EHEC by weight, with the balance comprising acetone. According to one exemplary embodiment, a biodegradable polymer comprises 5% EHEC, 5% PLGA, with the balance comprising acetone. Optionally, the biodegradable polymer further comprises a dye such as 2-FDCBL01 in a concentration of about 0.1% by weight. As yet another option, the water-soluble gel comprises a fluorosurfactant such as 3M Fluorad 1-FC4432 in a concentration of about 0.1-0.5% by weight, and more specifically, about 0.2% by weight.

G. Biodegradable Polymer Utilizing Polyoxypropylene-Polyoxyethylene Block Copolymer and Ethanol

According to one exemplary embodiment of the present application, the biodegradable polymer composition comprises polyoypropylene-polyoxyethylene block copolymer and ethanol. According to certain embodiments of the present application, a biodegradable polymer composition comprising comprises approximately 15%-50% polyoxypropylene-polyoxyethylene block copolymer by weight, and approximately 85%-50% ethanol by weight. Optionally, approximately 0.1%-6.0% of the composition may comprise water. Further optionally, approximately 0.001%-1.0% of the composition may comprise FD&C Red #40. According to one exemplary embodiment, a biodegradable polymer comprises approximately 40% polyoxypropylene-polyoxyethylene block copolymer, about 54% ethanol, about 6.0% water, and less than about 0.001% FD&C Red #40. It will be appreciated that poloxamer is a non-ionic substance having a balanced hydrophilic and hydrophobic structure unlike many of the hydrogels discussed above. Surprisingly, these properties show favorable results when used as a release agent, and still allow substantial ability to remove the release agent with water only, thereby allowing a strong bond with the dentin sealant such as All-Bond 2 or All-Bond 3 and the permanent restoration. As yet another option, the biodegradable polymer composition further comprises a fluorosurfactant such as 3M Fluorad 1-FC4432 in a concentration of about 0.1-0.5% by weight, and more specifically, about 0.2% by weight.

### Application

A. Initial Treatment of the Preparation Site

The water soluble or biodegradable polymers discussed above or below may be applied according to the following methodology. It will be appreciated that this is an exemplary embodiment of the application, and that other methods may be employed.

In employing the Immediate Dentin Sealing Technique, a practitioner thoroughly cleans the preparation site, and then etches the dentin for 15 seconds with an acid such as a phosphoric acid. UNI-ETCH® W/BAC is one such etchant available from Bisco, Inc. After etching, the preparation site is rinsed thoroughly and dried.

Optionally, using a brush or foam pellet or other applicator, the preparation site is moistened, disinfected, and cleaned with a solution of chlorhexidine digluconate such as CAVITY CLEANSER™ available from Bisco, Inc. Thereafter, a dental resin is applied to the prepared site. For example, one resin to be employed would include ALL-BOND 3™ from Bisco, Inc. In one embodiment, equal parts ALL-BOND 3™ Parts A and B are dispensed and mixed in a mixing well for 5 seconds. Immediately thereafter 1 to 2 coats of the resin are applied onto the prepared site, leaving the prepared surface shiny. If the surface is not shiny, additional coats are applied. Then, the resin surface is optionally air-dried for 10 seconds to evaporate the solvent. Thereafter, the dental resin is light cured for about 10 seconds at 500 mW/cm².

In the event that there is an undercut in the preparation site, the undercut should be blocked out the using a flowable composite such at AELITE-FLO™ according to manufacturer's instructions. While recontouring the composite surface to smooth the preparation, the enamel margin should be re-prepped. Thereafter, entire surface of the restoration should be clean and any oxygen-inhibited layer should be removed with an alcohol moistened cotton pellet or 2×2 inch gauze.

Thereafter, an impression of the prepared surface is made according to the impression material manufacturer's instructions. After the impression is created, the preparation is then thoroughly cleaned to remove any contaminants left from the impression process.

Next, a biodegradable polymer composition described above and/or below is applied to the prepared surface by dispensing I drop or another small amount of the biodegradable polymer composition described above and/or below into a mixing well. Using a brush, apply 1-2 coats of the biodegradable polymer composition to the entire preparation. Optionally, the practitioner may choose not to coat the margin of the preparation-approximately 1 mm-to assure retention of the temporary prosthetic that will be applied to the prepared surface. Further optionally, the biodegradable polymer is gently air dried for 20 seconds to evaporate the solvent.

B. Temporary Fillings or Provisional Inlays

For temporary fillings or provisional inlays, a practitioner would next administer the desired amount of a flowable dental resin or composite, such as PRO-V FLO™ available from Bisco, Inc., directly into the preparation. The composite or resin is then partially cured, for instance, by light curing the resin for 10 seconds at 500 mW/cm². Therafter, the practitioner would mark the occlusion and carve the prepared site, and allow the resin or composite to fully cure, for example by light curing the prepared site for an additional 20 seconds at 500 mW/cm².

As another option for temporary fillings or provisional inlays, instead of following the immediately previous paragraph, the practitioner may use a less flowable resin or composite, such as PRO-V FILL™ available from Bisco. The resin or composite may be applied in incremental layers of 2-3 mm, with each increment being light cured for approximately 20 seconds at 500 mW/cm². Once the desired height is achieved, the last incremental layer of resin or composite is light cure for about 10 seconds at 500 mW/cm². Thereafter, the practitioner would mark the occlusion and carve off the temporary prosthetic or inlay to fit. After the final carving, the practitioner would then light cure the entire prosthetic for an additional approximately 20 seconds at 500 mW/cm².

C. Provisional Onlays

If the practitioner wishes to create provisional onlays, after treatment of the preparation site as discussed in Example 1A, a resin or composite such as PRO-V FILL™ is built up in incremental layers of 2-3 mm, with each layer light cured for 20 seconds at 500 mW/cm². Once the desired height is achieved, the last incremental layer of resin or composite is light cured for about 10 seconds at 500 mW/cm². Thereafter, the practitioner would mark the occlusion and carve off the temporary prosthetic or inlay to fit. After the final carving, the practitioner would then light cure the entire prosthetic for an additional approximately 20 seconds at 500 mW/cm².

### D. Removal of Temporary Restoration

Once the final restoration has been created, and the patient returns to have the temporary restoration removed so that the final restoration may be placed over the preparation site, the practitioner will remove the temporary restoration by inserting a sharp instrument into the temporary restoration material and removing the restoration by applying pressure to allow the restoration to release from the preparation site occlusally. Thereafter, the practitioner will thoroughly clean the preparation site and continue with the permanent placement protocol for the final restoration.

If immediate dentin sealing was used, the practitioner should etch the enamel of the preparation site for 15 seconds with phosphoric acid such as UNI-ETCH® W/BAC. After etching, the preparation site is rinsed thoroughly and dried. Thereafter, a dental resin is applied to the prepared site. For example, one resin to be employed would include ALL-BOND 3™ from Bisco, Inc. In one embodiment, equal parts ALL-BOND 3™ Parts A and B are dispensed and mixed in a mixing well for 5 seconds. Immediately thereafter 1 to 2 coats of the resin are applied onto the prepared site, leaving the prepared surface shiny. If the surface is not shiny, additional coats are applied. Then, the resin surface is optionally air-dried for 10 seconds to evaporate the solvent. Thereafter, the dental resin is light cured for about 10 seconds at 500 mW/cm². Optionally, 1 thin coat of resin or composite is applied to the sealed dentin and light cured for 10 seconds at 500 mW/cm². Thereafter, cementation of the final preparation is performed according to manufacturer's instructions.

It will be appreciated that other methodologies may be employed, as the above method is presented for exemplary purposes.

### Testing

In order to test the above water soluble or biodegradable polymers, formulations that are representative of each of the above water soluble or biodegradable polymers were tested against control groups and commercial release coatings. In each of these tests, the relative shear bond strength of the temporary filling was tested against a control in which no release coating was utilized to determine whether the separating or releasing agent allowed for removal of the temporary restoration without leaving portions of the temporary restoration bonded to the tooth. For each of the tests, the following formulations were tested:

Tested Formulations

| Formulation | |
|---|---|
| No. | Chemicals (wt %) |
| 1 | 20% PVP in EtOH (“PVP”) |
| 2 | 20% PVP + 2% AR972 + 0.2% FC in EtOH (“PVP + |
| | R972”) |
| 3 | 10% PVP + 0.2% Agar in (EtOH + Water) (“PVP + Agar”) |
| 4 | 10% EHEC in EtOH (“EHEC”) |
| 5 | 10% PLGA in Acetone (“PLGA”) |
| 6 | 5% EHEC + 5% PLGA in Acetone (“PLGA + EHEC”) |
| 7 | Sun Medical SEP commercial separator (“SUN SEP”) |
| 8 | E-Z Sep commercial separator (Cosmedent, Inc.) |
| | (“E-Z Sep”) |
| 9 | Separator manufactured by Kerr (“Tempfil SEP”) |

### Comparative Shear Bond Strength: Pushout Test Results

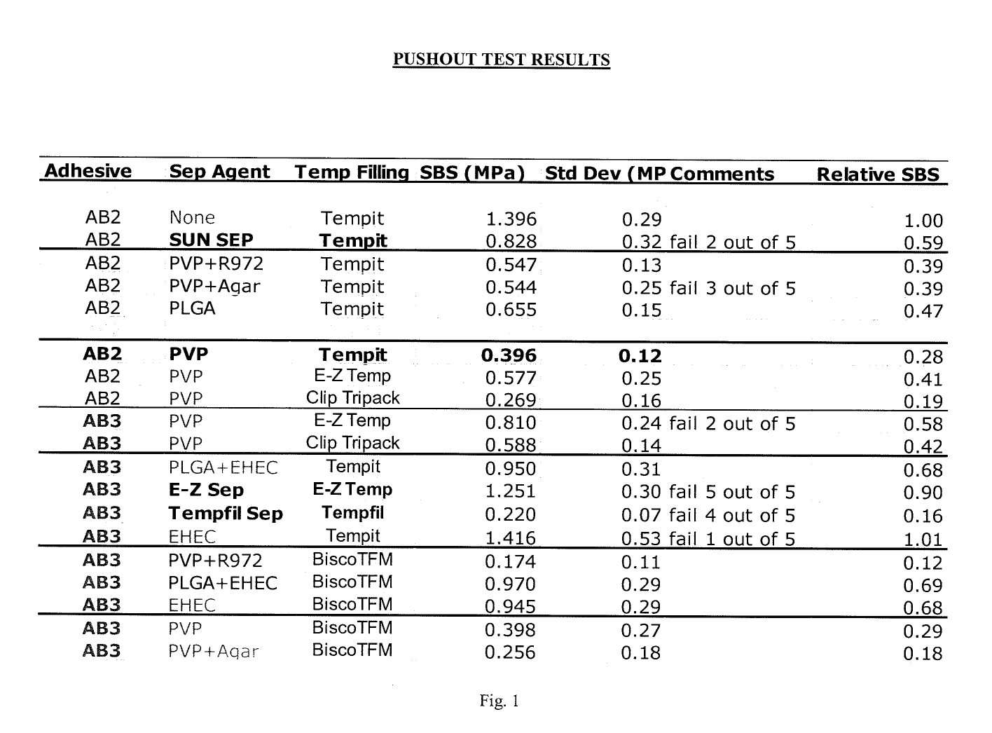

According to a first test, whose results are shown in FIG. 1, each of the formulations were tested with regard to the relative shear bond strength when the internal wall of a stainless steel hollow disc (I.D. 5.96×H. 3.00 mm) was treated with All Bond 3® (“AB3”) adhesive (Bisco), followed by the application of the respective separator and followed with a temporary filling material such as Tempit® moisture-activated temporary filling material (available from Centrix, Inc.), E-Z Temp® (available from Kerr), or Clip Tripack® temporary filling material (available from Voco, Germany) as noted. A pushout test was performed using a 10 kN load cell with an x-head speed of 5 mm/minute, with the relative shear bond strength results shown in FIG. 1. As noted in FIG. 1, a failure indicates an inability to remove the temporary filling in one piece due to adhesion of the filling material to the sealed surface. It will be appreciated that the lower relative SBS relates to better performance of the formulation, as this indicates relative ease of removing the temporary restoration.

### Comparative Shear Bond Strength: Pullout Test Results

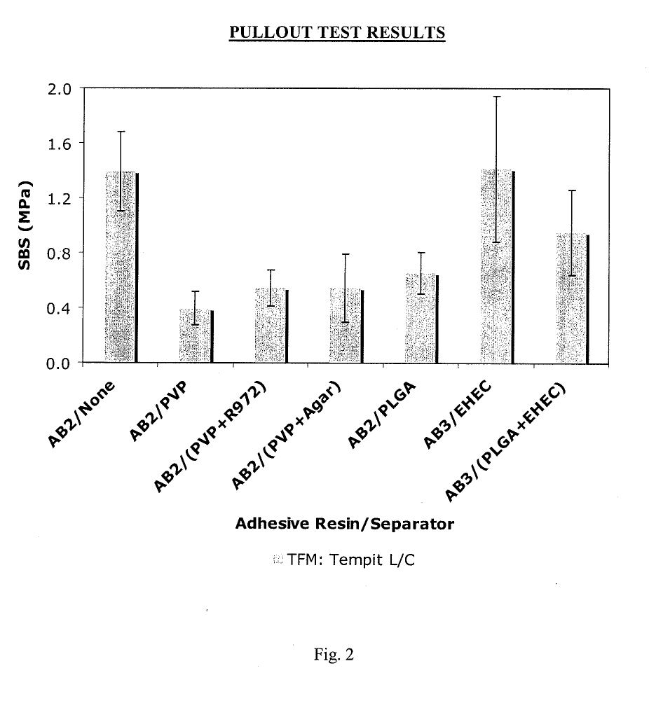

According to a first test, whose results are shown in a bar graph in FIG. 2, each of the formulations were tested with regard to the shear bond strength (measured in MPa) when a 3.5 mm×3.5 mm depression was drilled on a molar, and the tooth was treated in the IDS fashion by etching the prepared site with Bisco, Inc.'s Uni-Etch® etchant, rinsing the area after 15 seconds, and applying 1-2 coats of Bisco, Inc.'s All-Bond 2® (“AB2”) or All-Bond 3® (“AB3”) adhesive per the label directions to seal the dentin, and cured thereafter. Thereafter, a thin film separator was applied, with Tempit® moisture-activated temporary filling material (available from Centrix, Inc.). A pull test was performed using a 10 kN load cell with an x-head speed of 1 mm/second the relative shear bond strength results shown in FIG. 2. In order to allow the testing equipment to pull the temporary filling material from the cavity, an endodontic file was embedded in the temporary filling material, and the testing equipment was attached to the endodontic file to apply force thereon. No commercial products are compared in this test. It will be appreciated that the lower relative SBS relates to better performance of the formulation, as discussed above.

Further, turning now to Table 1 below, the following commercial separator formulations were compared to the polymer utilizing polyoxypropylene-polyoxyethylene block copolymer and ethanol (comprising 40% polyoxypropylene-polyoxyethylene block copolymer, 60% ethanol) using the pullout testing method discussed above. Failures are identified as tests in which the endodontic file pulled out of the temporary filling material while the temporary filling remained in the cavity.

TABLE 1
Pullout Test Results

| | Pullout Shear | Number |
|---|---|---|
| | Bond Strength | of |
| Materials Tested | (MPa ± SD) | Failure |
| AB3/E-Zsep/E-Z Temp | 1.251 ± 0.03 | 5/5 |
| AB3/TempfilSEP/Tempfil | 0.220 ± 0.07 | 4/5 |
| AB3/SB SEP/Tempit | 0.798 ± 0.29 | 2/5 |
| AB3/20% Polyoxypropylene- | 0.403 ± 0.19 | 0/5 |
| polyoxyethylene block | | |
| copolymer and water/Tempit | | |

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. For example, a time-release agent allowing leaching or degradation of the temporary separating material is anticipated as being a potential addition, as is the addition of micro-or nano-particles to any of the above formulations to increase thickness or viscosity of the formulation in instances where it is warranted.

What is claimed is:

1. A separating agent composition comprising:
a. a first non-ionic portion comprising a polymer comprising a hydrophobic and hydrophilic portion; and
b. a second portion comprising an alcohol.

2. The separating agent of claim 1, wherein the first portion comprises approximately 10% to approximately 50% by weight of the separating agent.

3. The separating agent of claim 1, wherein the first portion comprises approximately 35% to approximately 42% by weight of the separating agent.

4. The separating agent of claim 3, wherein the first portion is selected from the group consisting of polyoxypropylene-polyoxyethylene block copolymer and poly(lactic-co-glycolic acid).

5. The separating agent of claim 4, wherein the second portion is ethanol.

6. The separating agent of claim 5, further comprising water.

7. A temporary dental restoration adhesion system comprising:
a. a first dental resin operable to be applied to a prepared dental surface to seal said prepared surface, the dental resin comprising a hydrophobic portion;
b. a separating agent operable to coat a surface formed by the dental resin to act as a separation layer, the separating agent comprising a first non-ionic portion comprising a polymer comprising a hydrophobic and hydrophilic portion, and the separating agent further comprising a second portion comprising an alcohol; and
c. a second dental resin operable to be applied over the separation layer, the second dental resin comprising a hydrophobic portion such that the second dental resin is operable to form a bond with the hydrophobic portion of the separating agent.

8. The temporary dental restoration adhesion system of claim 7, wherein the separating agent further comprises water.

9. The temporary dental restoration adhesion system of claim 7, wherein the second dental resin is operable to form a removable bond with the separation layer such that the second dental resin, when cured, may be removed from the separation layer with less than 0.7 MPa pullout force.

10. The temporary dental restoration adhesion system of claim 7, wherein the second dental resin is operable to form a removable bond with the separation layer such that the second dental resin, when cured, may be removed from the separation layer with less than 0.5 MPa pullout force.

11. The temporary dental restoration adhesion system of claim 7, wherein the first portion of the separating agent comprises approximately 10% to approximately 50% of the separating agent by weight.

12. The temporary dental restoration adhesion system of claim 7, wherein the first portion of the separating agent comprises approximately 35% to approximately 42% of the separating agent by weight.

13. The temporary dental restoration adhesion system of claim 7, wherein the first non-ionic portion of the separating agent is selected from the group consisting of polyoxypropylene-polyoxyethylene block copolymer and poly(lactic-co-glycolic acid).

14. The temporary dental restoration adhesion system of claim 7, wherein the first non-ionic portion of the separating agent comprises microparticles.

15. The temporary dental restoration adhesion system of claim 7, wherein the first non-ionic portion of the separating agent comprises nanoparticles.

16. The temporary dental restoration adhesion system of claim 7, wherein the second portion of the separating agent is ethanol.

17. The temporary dental restoration adhesion system of claim 7, wherein the second portion further comprises water.

18. A temporary dental restoration adhesion system comprising:
a. a first dental resin operable to be applied to a prepared dental surface to seal said prepared dental surface, the dental resin comprising a hydrophobic portion;
b. a separating agent operable to coat a surface formed by the dental resin to act as a separation layer, the separating agent comprising a first portion comprising polyoxypropylene-polyoxyethylene block copolymer having a hydrophobic and hydrophilic portion, and the separating agent further comprising a second portion comprising ethanol, wherein the first portion comprises approximately 10% to approximately 50% of the separating agent by weight; and
c. a second dental resin operable to be applied over the separation layer, the second dental resin comprising a hydrophobic portion such that the second dental resin is operable to form a bond with the hydrophobic portion of the separating agent.

19. The temporary dental restoration adhesion system of claim 18, wherein the separating agent further comprises water.

20. The temporary dental restoration adhesion system of claim 19, wherein the polyoxypropylene-polyoxyethylene block copolymer is dispersed throughout the separating agent as nanoparticles.
